(19) United States
(12) Patent Application Publication
(10) Pub. No.: US 2007/0161869 A1
(43) Pub. Date: Jul. 12, 2007

(54) Liquid or semi-liquid movement and or dispensing system used for entertainment or health care purposes over a global communications network

(76) Inventor: Michael Goudie, Sunnyvale, CA (US)

Correspondence Address: MICHAEL GOUDIE, 966 PONDEROSA AV - 56, SUNNYVALE, CA, 94086, US

(21) Appl. No.: 11/329,880

(22) Filed: Jan. 12, 2006

(51) Int. Cl. A61M 31/00

(52) U.S. Cl. 600/300; 604/500

(57) ABSTRACT

A system that controls the movement of liquid(s) or semi-liquid(s) for entertainment or health care purposes over a global communication network controlled by hardware or software. This system can be connected to other similar systems and used as an intermediary to promote the movement of liquid(s) or semi-liquid(s), or it can be used as a stand-alone system to dispense the liquid(s) or semi-liquid(s) controlled over a global communications network for entertainment or health care purposes.

### FIELD OF THE INVENTION

This invention relates the movement of liquid(s) or semi-liquid(s) for entertainment or health care purposes, in particular it relates to the movement of liquid(s) or semi-liquid(s) over a global communication network controlled by human or machine generated input for entertainment or health care purposes. This invention describes how a mechanical apparatus can be used with the following input: human to human, human to machine, machine to human or any group of the preceding. A global communication network can be comprised of a group of computers connected to each other communicating by sending and receiving data packets. Such a network is referred to today as the Internet. Another form of a global communication network is a cell phone system where a human or machine can communicate to a cell phone or machine.

### BACKGROUND OF THE INVENTION

The movement of liquid(s) or semi-liquid(s) can be used for entertainment or health care purposes between entities that interact with each other over a global communications network. Entertainers and are seeking new ways to entertain and health care professionals are looking for new ways to administer health care over a global communications network. This invention will describe how this novel new machine can provide great entertainment value to entertainers at large, and how it can provide health care. This apparatus is controlled over a global communications network like the Internet and can be used in conjunction with live radio, or television broadcasts to allow for greater audience participation for entertainers, or health care professionals whom administer health care can use it in conjunction with a live broadcast to oversee healthcare.

### SUMMARY OF THE INVENTION

It is an object of the invention to provide an apparatus used for the movement of liquid(s) or semi-liquid(s) controlled over a global communications network for entertainment or health care purposes.

The invention claimed herein is a method, software, and hardware to allow for the movement of liquid(s) or semi-liquid(s) over a global communications network for entertainment or health care purposes. Users can be mechanical or human. A control signal is generated by machine, software, or software interface and sent over a global communications network. A machine that converts or directly passes the control signal to the liquid moving apparatus receives this control signal, or the apparatus is connected directly to the global communications network and the apparatus directly processes the control signal. The connection to the global communications network can utilize a hard wire or wireless connection. The liquid movement and or dispensing apparatus receives packets of data that it consumes in responds to control signals sent to it from a network communication tool such as a cell phone, PDA, computer, or machine. The apparatus can have it's own processor and communication protocol so it can be directly connected to a communication network.

The system in claim 1 allows a user to connect the apparatus as described, to a global communication network through a computer or thought a network interface designed to communicate with other devices. This interface can be a built in feature of the apparatus itself.

The system in claim 1 can consist of a single or group of the liquid movement and or dispensing apparatus' that are able to communicate over a communication network. It is the intention of the inventor to obtain a patent for a single or plurality of liquid movement and or dispensing apparatus' that are able to communicate over a communication network.

The apparatus introduced in claim one has an internal processing mechanism used for the movement of liquid(s) or semi-liquid(s). This internal mechanism is described herein as a motorized pump that is connected to a controlling circuit board and a fluid reservoir. There may be different configurations to the processing mechanism to someone skilled in the art of fluid dynamics. Such alternating configurations could include the use of compressed air, solenoid valves controlled by a controlling circuit, and a fluid reservoir. The may be many different configurations used to process the movement of the liquid(s) or semi-liquid(s). I will illustrate the use of a motorized pump, but this is by no means the only way to move the liquid(s) or semi-liquid(s).

The apparatus uses a motorized pump to effectively move or dispense the liquid(s) or semi-liquid(s). This motorized pump is comprised of a motor and a pump that is used to move liquid(s) or semi liquid(s). This motorized pump is controlled by a controlling circuit that turns the motor on when it receives a command signal to do so. The command signal in this example is received via a USB connection to a computer. That computer is itself connected to the Internet and uses software to receive a control signal sent to it from an originating or relaying source. That software can translate the control signal or it can directly send it to the controlling circuit in the apparatus. Again, this is an example to describe an overall process, someone skilled in the art of global communication design may process the signal a little differently based on hardware, network, or apparatus configurations.

The apparatus has a reservoir or a connection to a source of liquid(s) or semi-liquid(s). When the controlling circuit activates the motorized pump it draws liquid(s) or semi-liquid(s) from the reservoir and moves them or dispenses them from the apparatus.

The movement or dispensing of the liquid(s) or semi-liquid(s) is used for entertainment or health care purposes. One such entertainment example may be a television show that squirts goo all over the game show contestants as they participate in a game. The producers of the show can now allow greater audience participation by allowing the goo to be shot over the game contestants by the home viewing audience over the Internet. A healthcare example may be the dispensing of medicines. The apparatus might have a reservoir of liquid medicine that the health care professional administers remotely so patients don't forget to take their medication. These are just a few examples and by no means the only usage of this technology in the entertainment, or health care fields.

### BRIEF DESCRIPTION OF THE DRAWINGS

FIG. 1 is a labeled drawing of an apparatus that moves or dispenses liquid(s) or semi-liquid(s) for entertainment or health care purposes;

FIG. 2 is a labeled drawing of the apparatus connected to a global communications network using a personal computer.

FIG. 3 depicts operation of the system according to the preferred embodiments.

### DETAILED DESCRIPTION OF THE PREFERRED EMBODIMENTS

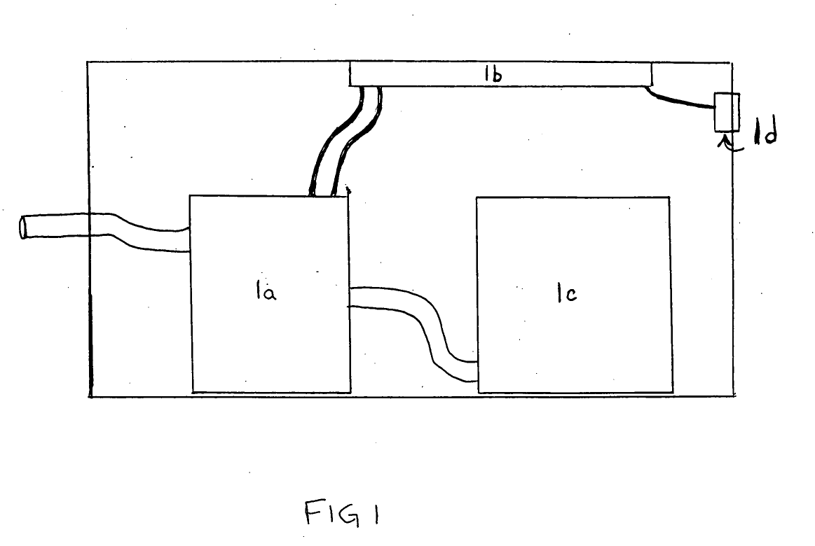

Referring to FIG. 1, this is a system that moves liquid(s) or semi-liquid(s) for entertainment or health care purposes over a global communication network. Inside the apparatus is a motorized pump (1a), a printed circuit board control circuit (1b), a reservoir (1c) for storage of liquid(s) or semi-liquid(s), and a connector (1d) used to connect the control circuit to a computer that is connected to a global network.

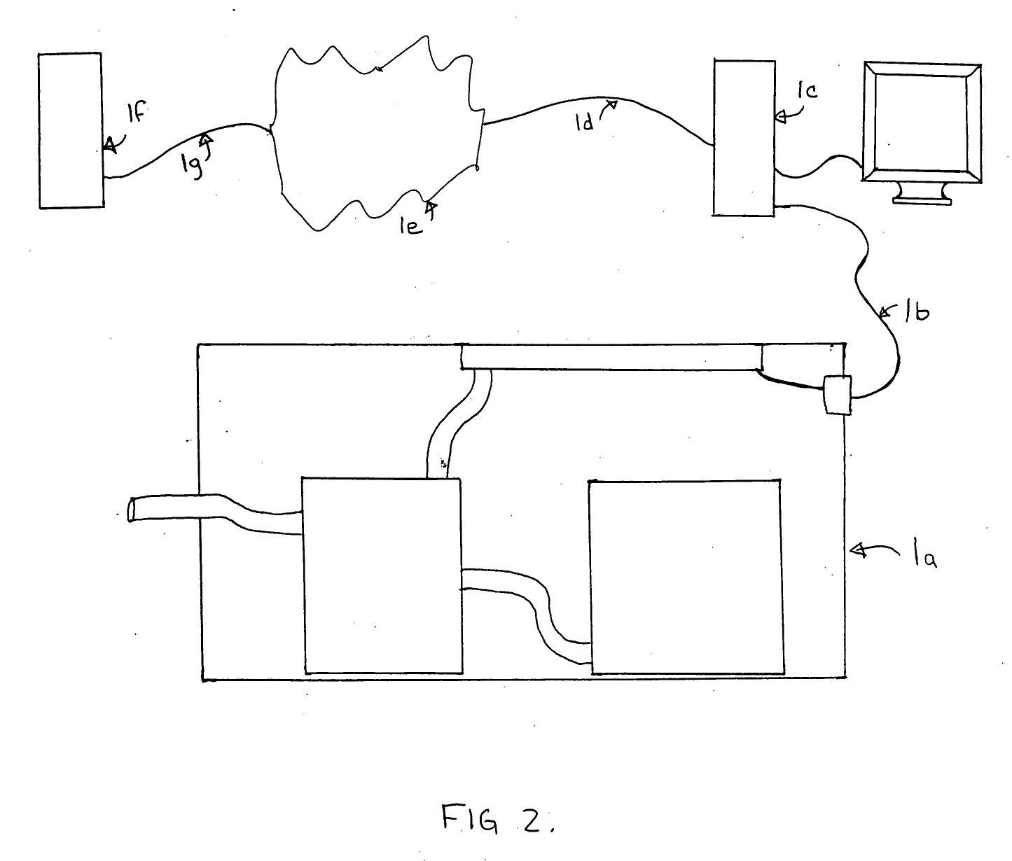

FIG. 2 is a depiction of the system connected to a global network. The system can be comprised of a single apparatus (1a) that moves liquid(s) or semi-liquid(s) for entertainment or health care purposes over a global communication network, or a plurality of apparatuses that move liquid(s) or semi-liquid(s) for entertainment or health care purposes over a global communication network.

The apparatus (1a) is connected to a computer (1c) via a USB cable (1b). The computer (1c) is connected to a global communications network (1e) via a network cable (1d).

A computer (1f) that generates a control signal is connected to a global communications network (1e) via a network cable (1g). Any device that can communicate over the global communications network can generate the control signal.

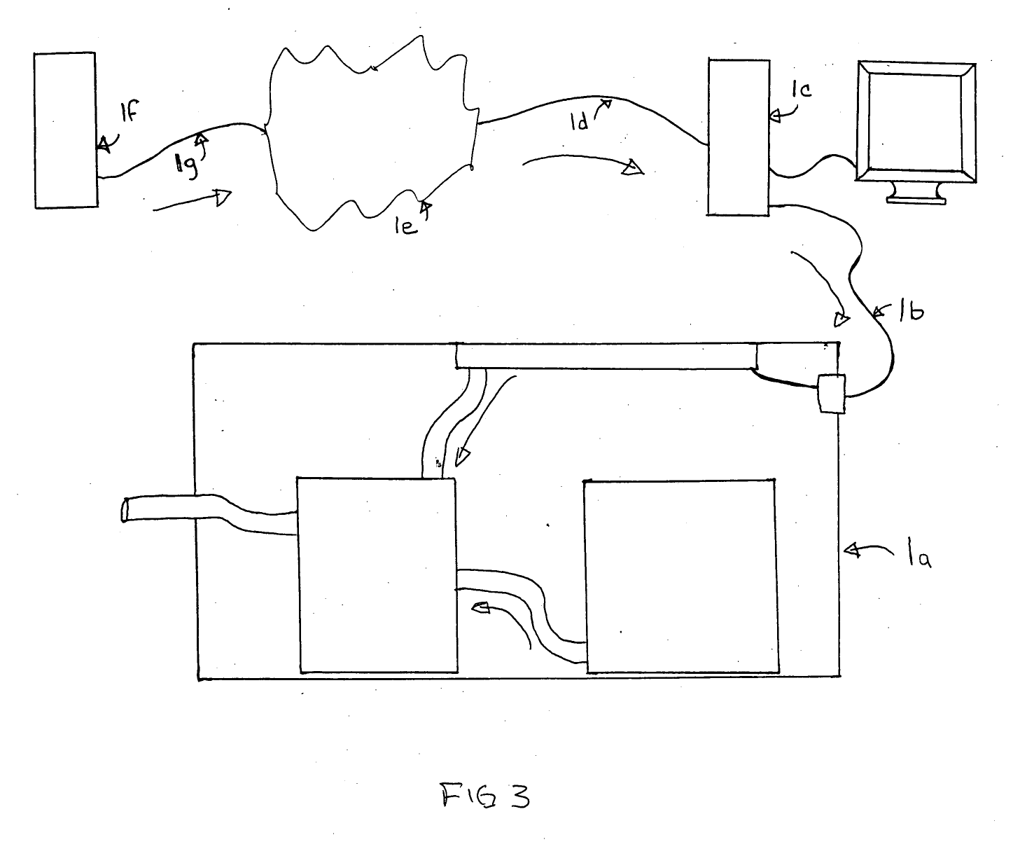

FIG. 3. The drawing is a description of how the flow of communication can work. This is not the only way, rather one way used to convey the general concept.

In this depiction the computer (1f) generates a control signal that is sent over the global communications network (1e). That control signal is then routed to the receiving computer (1c). The receiving computer (1c) then processes the control signal and sends it to the apparatus that moves liquid(s) or semi-liquid(s) for entertainment or health care purposes. The circuitry in the apparatus then consumes the signal and makes the motor move that in turn activates the pump and draws material from the reservoir. That action then moves liquid(s) or semi-liquid(s) or dispenses the liquid(s) or semi-liquid(s). The apparatus can then send signals back to the receiving computer (1c), that in turn can communicate back to the originating computer (1f), or the apparatus can communicate to another apparatus, or some other configuration of communications.

1. A system that moves liquid(s) or semi-liquid(s) for entertainment or health care purposes over a global communication network comprising the following:
An apparatus that has internal components that allow for the movement and dispensing of liquid(s) or semi-liquid(s) that is activated when a control signal is sent to it over a global communications network. The control signal is generated by software or hardware in response to human interaction, or machine interaction.

2. The system in claim 1 consists of an apparatus that has internal circuitry that controls mechanical components that assist in the movement and or dispensing of liquid(s) or semi-liquid(s) for entertainment or health care purposes. The internal circuitry of the apparatus referred to in claim one can be designed as a stand-alone circuit that has it's own tcp/ip stack or other protocol communications stack and processor(s) that connects itself to a global communication network hard-wired or wirelessly, and can process or create control signals, or the apparatus referred to in claim one can be designed to create or process a control signal that is generated or passed to it via a hard-wired or wireless connection from a machine that has the ability to communicate over a global or has the ability to create the control signal that is used by to apparatus to control the movement of liquid(s), or semi-liquid(s) for entertainment or health care purposes.

3. The apparatus in claim one is used to move and or dispense liquid(s) and or semi-liquid(s) for entertainment or health care purposes, as an end point or as a intermediary devise. As an end point, the apparatus in claim one dispenses liquid(s) or semi-liquid(s), as an intermediary the apparatus in claim one promotes the movement of liquid(s) or semi-liquid(s) for entertainment or health care purposes but does not dispense the liquids(s) or semi-liquid(s).
